(19) United States
(12) Patent Application Publication
(10) Pub. No.: US 2006/0099245 A1
(43) Pub. Date: May 11, 2006

(54) Hydrodynamically balancing oral drug delivery system with biphasic release

(76) Inventors: Manoj Kumar, Haryana (IN); Naresh Talwar, New Delhi (IN); Rajeev Singh Raghuvanshi, New Delhi (IN); Ashok Kumar Rampal, Punjab (IN)

Correspondence Address: RANBAXY INC., 600 COLLEGE ROAD EAST, SUITE 2100, PRINCETON, NJ, 08540, US

(21) Appl. No.: 10/514,674

(22) PCT Filed: May 21, 2002
(86) PCT No.: PCT/IB02/01739

§ 371 Date: Sep. 2, 2005

Related U.S. Application Data
(63) Continuation-in-part of application No. 10/048,870, filed on Apr. 15, 2002.

(51) Int. Cl. A61K 9/48; A61K 9/64; A61K 9/22; A61K 31/403

(52) U.S. Cl. 424/451; 424/468; 514/411; 424/456

(57) ABSTRACT

The present invention relates to an oral drug delivery system with biphasic release characteristics comprising a porous matrix comprising at least one drug substance, sugar(s), a release retarding polymer, gas generating components and optionally, pharma-ceuti-cally acceptable auxiliary components wherein the pharmaceutical composition further comprises a coating of said drug substance. The pharmaceutical composi-tion, either in the form of pellets (multiparticulate or single unit dosage form), beads, granules, capsules or tablets, is retained in the stomach while selectively delivering the drug(s) at gastrointestinal levels and upper parts of the small intestine over an extended period of time. The release of the drug from the said pharmaceutical composition is characterized by a biphasic release profile of the drug substance, which exhibits both immediate and controlled release characteristics.

### FIELD OF THE INVENTION

The present invention relates to an oral drug delivery system with biphasic release characteristics comprising a porous matrix comprising at least one drug substance, sugar(s), a release retarding polymer, gas generating components and optionally, pharmaceutically acceptable auxiliary components wherein the pharmaceutical composition further comprises a coating of said drug substance. The pharmaceutical composition, either in the form of pellets (multiparticulate or single unit dosage form), beads, granules, capsules or tablets, is retained in the stomach while selectively delivering the drug(s) at gastric levels and upper parts of the small intestine over an extended period of time.

### BACKGROUND OF THE INVENTION

An orally administered drug delivery system is exposed to a wide range of highly variable conditions, such as pH, agitation intensity, gastric emptying times and composition of the gastrointestinal fluids during its transit through the digestive tract. In addition, presence of food in the tract may affect the dosage form performance. Therefore, to design an optimum oral controlled release system it is necessary to take into account the physico-chemical and physiological environment of the gastrointestinal tract. The conventional approaches to controlled release formulation known in the art are not applicable to a variety of drugs having an “absorption window” in the stomach or upper parts of small intestine. Furthermore, it is advantageous to retain the dosage form in the stomach thereby increasing the contact time for local activity and to achieve better therapeutic efficacy for the diseases which are confined to the upper parts of the gastrointestinal tract such as peptic and duodenal ulcers.

It is readily apparent that a sustained release formulation that slowly releases medicament over an extended period and is retained in the upper parts of gastrointestinal tract for a prolonged period would be desirable for such diseases.

The prior art discloses various approaches for therapeutic dosage forms which are designed to be retained in the upper parts of the gastrointestinal tract and possess sustained release characteristics.

U.S. Pat. No. 5,780,057 discloses a pharmaceutical tablet having a multilayer structure wherein at least one layer swells in the presence of biological aqueous fluids resulting in an increase by at least 50% of the total volume of the tablet and thereby allegedly exhibiting a high residence time in the stomach and/or in the upper portion of the gastrointestinal tract. The swellable layer, being a granular mixture of biocompatible hydrophilic polymers and highly swellable (super disintegrating) polymers, allegedly acts as a barrier and allegedly modulates the slow release of the active ingredient from the pharmaceutical form. It is believed that the expanded dosage forms could block the pyloric sphincter or could cause unfavorable conditions following multiple dosing resulting from retention of swollen dosage units in the stomach.

U.S. Pat. No. 5,651,985 discloses a composition comprising 30-90%, by weight of the composition, a homogenous mixture of polymers containing lactam groups and polymers containing carboxyl groups as gel forming agents, which swells to form a gel of allegedly high mechanical and dimensional stability in the aqueous environment of the stomach. It is believed that as the concentration of the polymers is very high, the dosage forms containing a high dose medicament would be large and inconvenient for oral administration.

U.S. Pat. No. 5,007,790 discloses a sustained-release oral drug dosage form comprising a plurality of solid particles of a solid-state drug dispersed within a hydrophilic, water swellable polymer that swells on imbibition of gastric fluid to increase the particle size to a level that promotes retention in the stomach over said time period, permitting dissolution of the dispersed drug and release of the resulting solution through a leaching action. The swellable polymer also allegedly maintains its physical integrity for at least a substantial portion of the time period during which the drug is released into the stomach and thereafter, rapidly dissolves. It is well recognized by those skilled in the art that it may be difficult to obtain the desired rate of release for a drug that has a high water solubility from such multiparticulate systems as described in this patent, in which the drug first undergoes dissolution followed by release of the resulting solution by leaching action.

U.S. Pat. No. 5,169,638 discloses a buoyancy controlled release powder formulation for releasing a pharmaceutical of a basic character regardless of the pH of the environment and which formulation includes upto about 45% by weight of a pH dependent polymer which is a water soluble salt of a polyuronic acid and upto about 35% by weight of a pH independent hydrocolloid gelling agent having a viscosity from about 50 to about 100,000 centipoises in a 2% solution at 20° C. The said formulation allegedly floats in the gastric fluid and release the drug at a controlled rate irrespective of the pH of the environment. However, the invention is particularly adapted for release of medicaments of only basic nature. Acidic drugs are not amenable for this system.

U.S. Pat. No. 4,814,179 discloses a floating, sustained release therapeutic composition in form of a non-compressed tablet having a network of multitudinous air holes and passages therein and a density of less than one comprising a matrix containing 0.5-4% gelling agent, 10-20% oil, 50-75% therapeutic agent and water. As exemplified therein, the preparation of non-compressed tablet requires unconventional processing techniques and uses molds with cylindrical holes for the same. This involves manufacturing difficulties and is cost enhancing too.

U.S. Pat. No. 4,702,918 discloses a floating, sustained release formulation formed by heating a mixture of a gelling agent (cellulose or starch derivative) and a fat/oil which is solid at room temperature. A sustained-release capsule dosage form as disclosed therein contains a mixture of (a) from about 10 to about 90% by weight of a cellulose derivative or a starch derivative which forms a gel in water and (b) from about 90 to 10% by weight of a higher fatty acid glyceride or higher alcohol or a mixture thereof which is solid at room temperature and (c) from 0.01 to about 85% by weight of a pharmaceutical. The capsules are prepared by filling with said mixture of (a), (b) and (c), heating to a temperature above the melting point of fatty acid glyceride or higher alcohol and cooling and solidifying said mixture. More than mere mixing is required to impart buoyancy to the formulation, i.e., melting followed by cooling are additional unit operations. The specific gravity of digestive fluids especially that of gastric juices is between 1.004 to 1.101. It is well known to those skilled in the art that it may be difficult to maintain the low specific gravity for the sustained release composition as described in this patent, for a prolonged period. Therefore, the chances of such a system failing to release the drug in a sustained manner is relatively high.

U.S. Pat. No.4,126,672 discloses formulations comprising one or more medicaments in combination with a hydrocolloid or mixtures of hydrocolloids so as to have a bulk density less than one and be hydrodynamically balanced when in contact with gastric fluid. A sustained release capsule dosage form as described therein comprises finely particulate, homogenous mixture of chlordiazepoxide and diazepam, about 5% to 60% by weight of therapeutically inert, pharmaceutically acceptable adjunct materials, about 0% to 60% by weight of a fatty material having a specific gravity of less than one and about 20% to 75% by weight of one or a mixture of hydrocolloids selected from the group consisting of methyl cellulose, hydroxypropyl cellulose hydroxypropyl methylcellulose, hydroxymethyl cellulose and sodium carboxymethyl cellulose. Upon contact with gastric fluid, the hydrophilic colloid hydrates and this hydrated layer allegedly thereafter slowly dissolves to release the medicament. The release of medicament is also said to take place by leaching action at or near the surface. The hydrated colloid allegedly forms an outside barrier which retains the shape of the capsule and therefore acts to prevent the mass from disintegrating. However, it is well recognized that the application of such a system to obtain the desired rate of release of the drug wherein it is regulated by the erosion of the polymer, is difficult to maintain.

For the above stated reasons and because the prior art discloses either complicated devices and systems which are difficult to manufacture on the industrial scale or the components used therein are not so user friendly, none of the oral controlled drug delivery systems described heretofore is completely satisfactory.

U.S. Pat. No. 6,261,601 describes a pharmaceutical composition in the form of tablets or capsules, which provides a combination of spatial and temporal control of drug delivery when ingested by a patient. The pharmaceutical composition constitutes an oral controlled drug delivery system, comprising a drug, a gas generating component, a swelling agent, a viscolyzing agent and optionally a gel forming polymer. The viscolyzing agent and the gel forming polymer form a hydrated gel matrix which entraps the gas, causing the tablet or capsule to retain in the stomach or upper part of the small intestine (spatial control) and also creates a tortuous diffusion path for the drug, resulting in sustained release of the drug (temporal control). However, such formulations have been found to be unsuitable for formulations containing less than 35% w/w of the active ingredient.

Therefore, there is a need to have formulations in which the active ingredient constitutes 35% or less of the total weight providing a predictable and uniform treatment regimen for oral administration and which have the further advantage of simplifying treatment and improving patient compliance while both enhancing the bioavailability of the active ingredient and prolonging the release of the drug.

Co-pending PCT application PCT/IB00/0183 published as WO 01/10419 describes a pharmaceutical composition in the form of an oral drug delivery system for prolonged gastric retention comprising a highly porous matrix comprising at least one drug substance, sugar(s), gas generating components and optionally, pharmaceutically acceptable auxiliary components.

The invention is directed to compositions structurally composed of a buoyant honeycombed matrix with enhanced gastric retention that selectively releases the drug in a controlled manner over a prolonged period of time. However, it is well recognized by those skilled in the art that the polymeric systems so designed for sustained or controlled drug delivery function on the different release mechanism such as dissolution, erosion, diffusion and the like. Such systems are custom designed to slow the release of the drug from the delivery system. Such a system warrants a lag time, which refers to the duration of time between the administration of the composition and the delivery of drug substance from the same. This lag results in delayed availability of the drug for the immediate therapeutic action. Such delivery systems are therefore not suitable for treatment of ailments that require immediate attention in addition to continuous therapy.

Further, in order to efficiently release the site-selective delivery of a medicament to the stomach, it is necessary to design an optimal oral controlled release system taking into account the physio-chemical and physiological environment of the gastrointestinal tract. The gastric residence time is subject to a very significant inter-individual variations and is, inter alia, dependent on the nutritional habits of the individual. Meals of high calorific value, especially fats, have an inhibitory effect on gastric emptying. However, upon oral administration the drug formulation usually traverses the stomach in about 3-4 hours which may be increased further using gastroretentive delivery systems as is taught by WO 01/10419.

Drugs having an “absorption window” in the stomach and upper gastrointestinal tract may not be completely absorbed when administered in the form of a typical oral controlled drug delivery system. Slow release formulations of such drugs may only be effective for about 4-5 hours whereafter the formulation passes into the colon and the drug absorption reaches the minimum.

An initial lag time especially in the specific absorption window evidently hampers the total effective absorption of the drug. The means for achieving retention of drug formulations in the proximal region of the gastrointestinal tract, and for controlled release affecting therapeutic efficacy of such drugs therein has been a long sought objective.

Oral controlled release delivery systems should ideally be adaptable so that release rates and profiles can be matched to physiological and chromo-therapeutic specifications.

It has now been discovered that the pharmaceutical compositions described in WO 01/10419 when coated with the drug substance exhibit biphasic release characteristics that meet the requirements of both immediate and continuous medication. The drug coating provides the initial pulse of the release profile that accounts for the immediate therapy while the drug entrapped in the porous matrix exhibits sustained action.

The biphasic release allows the formulator to compensate for the lag period upon administration changing absorption rates of the drug in the gastrointestinal tract by providing a rapid onset of action and compensate for relatively slow absorption by providing a relatively controlled release rate.

The principle of sustained release which characterizes the formulations of the subject invention is unique in the art and no teaching has been found which recognizes the application of such a porous matrix to buoyancy and biphasic release as is taught by the present invention.

### SUMMARY OF THE INVENTION

It is an object of the present invention to provide a pharmaceutical composition in the form of pellets, beads, granules, capsules or tablets which constitutes an oral controlled drug delivery system that:

(a) provides biphasic release profile of the drug substance that exhibits both immediate and controlled release characteristics,

(b) constitutes a coating of the drug substance that provides initial pulse for rapid onset of action and a polymeric matrix that exhibits controlled release in the later phase,

(c) generates a gas to form a porous (preferably honeycombed) matrix with good floating characteristics and also evolves gas upon contact with gastric fluid which helps in retaining the buoyancy of the dosage form in the stomach,

(d) provides increased gastric residence and thereby extends residency of the drug delivery system in the gastrointestinal tract,

(e) delivers the drug at a controlled rate and exhibits reproducibility of release rate into aqueous media while floating in the stomach, and

(f) provides, as compared to other oral controlled drug delivery systems, increased absorption of a drug that is absorbed largely from the upper parts of the gastrointestinal tract.

It is also an object of the present invention to provide a pharmaceutical composition constituting an oral controlled drug delivery system that maintains its physical integrity and dimensional stability when in contact with gastric fluids. The system remains floating in-vitro in the simulated gastric fluid till substantially all the drug is released.

The present invention describes a therapeutic system either in the form of beads, pellets, or granules filled in a capsule (multiparticulate system) or single unit pellets and matrix capsules/tablets (monolithic system) which constitutes an orally administered buoyant delivery system capable of extended retention in gastrointestinal fluids. The delivery system is structurally composed of a porous matrix (preferably honeycombed) with large volume of entrapped air which makes it light and imparts good floatation characteristics, and a coating of drug substance that provides immediate release for rapid onset of action.

The therapeutic system comprises drug, sugar, gas generating components and optionally, pharmaceutically acceptable auxiliary components and a coating of said drug substance.

The gas generating components used herein are a combination of atleast one thermostable and atleast one thermolabile agent. During the preparation of formulation, on exposure to high temperature, the thermolabile agent generates gas and aids in attaining the porous internal structure, while the thermostable agent reacts with acidic gastric contents of the stomach to evolve gas which helps in maintaining buoyancy of the dosage form. Thus, the combination of gas generating components permits the therapeutic system to act as a floating matrix that extends the retention of the dosage form in the stomach and also prolongs its release in the stomach and upper parts of the small intestine. That is, the system is not transported past the “absorption window” prior to releasing all or substantially all of the drug and maximum bioavailability is attained.

Preferably, the oral controlled drug delivery system of the present application which is in the form of multiparticulate or a monolithic system, comprises an amount ranging from a pharmaceutically acceptable amount up to 35% of the total drug, of which about 5% to about 60% by weight of the drug may be present as active coating, about 5% to about 90% by weight of a sugar, about 1% to about 40% by weight of the gas generating components and, pharmaceutically acceptable auxiliary components.

### DETAILED DESCRIPTION OF THE INVENTION

According to the present invention, the oral pharmaceutical composition includes at least one drug substance, sugar(s), a combination of gas generating agents and optionally other pharmaceutical auxiliary components which may be used by one skilled in the art to formulate the therapeutic system. The choice of auxiliary components and the amounts to be used is considered to be within the purview of one skilled in the art. It is to be borne in mind, however, that these conventional pharmaceutical auxiliary components which might adversely affect the hydrodynamic balance of the formulation of the present invention are not suitable for use therein.

The gas evolved during the preparation of the formulation by the gas generating components causes the system to attain a porous structure. The drug is incorporated within this porous, preferably honeycombed matrix.

The composition may be in the form of pellets, beads, granules filled within a capsule or a sachet (a multiparticulate drug delivery system) or matrix capsules/tablets and single unit pellets (monolithic system) which are coated with the drug. The art of producing spherical pellets by extrusion and spheronisation techniques or spheronisation using techniques based on high shear granulation or fluidized bed techniques is well known and may be used for the preparation of pellets, beads or granules in the subject invention. Single unit pellets can be produced on industrial scale using lozenge and troches cutting machines.

Drugs which are thermostable may be added into the matrix while thermolabile drugs can be loaded onto the carrier spheres (drug free pellets) using techniques of drug loading based on fluidized bed principle (equipments like Glatt) which are well known in the art. The pharmaceutical composition of the present invention may be in the form of a multiparticulate drug delivery system (up to 4 mm in size pellets, granules or beads) or a single unit form as matrix capsule/tablet or large size pellets (more than 5 mm in size). The matrix capsule of the present invention may be produced by filling the powder according to the invention in a capsule made up of either gelatin, starch or hydroxypropyl methylcellulose followed with heat treatment.

Additional polymers recognized in the art of pharmaceutical compounding for their release retarding properties may also be incorporated into the formulation of the present invention. These release retarding polymers may be hydrophilic or hydrophobic in nature or may be pH dependent or independent polymers. Examples of the polymers suitable for this invention include hydroxypropyl methylcellulose, hydroxypropyl cellulose, Eudragit, ethyl cellulose, xanthan gum, and the like.

The pharmaceutical composition of the present invention is coated with the drug substance that provides the initial pulse of biphasic release for rapid onset of action. Further, the pharmaceutical composition may be coated with a film forming polymer to control the release of the drug or to impart better/improved floating characteristics (which is a result of better entrapment of the gas) or to improve its organoleptic properties. Furthermore, the pharmaceutical composition may also contain bioadhesive polymers incorporated within the coating or present as a film coat on the pellets, granules, beads, capsules or tablets in order to improve its gastro-retentive properties. In another application, some highly swelling polymers may also be added to increase the size of the dosage form so as to improve its gastric retention.

The pharmaceutical composition of the subject invention, when added to simulated gastrointestinal fluids, floats on the fluid till substantially all the drug is released. The thermostable gas generating agent included therein reacts with the acid present in the media and generates gases which become entrapped within the matrix thereby enhancing the buoyancy of the formulation.

The various components of the present invention are described in more details below.

Drug

According to the present invention, the pharmaceutical composition is in the form of pellets, beads or granules filled in a capsule, a matrix capsule/tablet or a matrix pellet, as a single unit that provides biphasic release that encompasses immediate release followed by controlled release of at least one therapeutic agent or drug. The drug may be pharmacologically active itself or may be converted into the active form by biotransformation in the body. The drug can be any drug for which therapy would be improved as a result of biphasic drug delivery and increased gastric retention.

The medicament or combination of medicaments which are amenable to biphasic release therapy utilising the novel formulations of the present invention include any of those suitable for oral administration. The present invention is not to be construed as being limited to any particular medicament or class of medicaments.

The formulations of the subject invention are particularly amenable to the administration of medicaments which are predominantly absorbed through the upper portion of the gastro intestinal tract, drugs having pH dependent solubility, i.e., more soluble in the gastric pH as compared to the intestinal pH, drugs having stomach as a site of action which includes H-2 receptor antagonists, antacids, antimuscarinic agents, proton pump inhibitors, drugs active against H. pylori, cytoprotective agents, and the like.

Illustrative examples of drugs that are absorbed predominantly from the upper parts of gastrointestinal tract include ciprofloxacin, cyclosporin, furosemide, metoprolol, oxprenolol, baclofen, allopurinol, sumatriptan, benazepril, enalapril, quinapril, moexipril, indolapril, olindapril, retinapril, spirapril, clilazeprilat, lisinopril, imidapril, benazeprilat, cilazapril, captopril, delapril, tosinopril, libenzapril, pentopril, perindopril, altiopril, quinaprilat, ramipril, spiraprilat, zofenopril, and the like; all of which are suitable for use in the present invention.

Drugs having the stomach as site of action include H-2 receptor antagonists such as ranitidine, famotidine, nizatidine, bifentidine, erbrotidine, nifentidine, roxatidine and cimetidine, and the like; proton pump inhibitors like omeprazole, lansoprazole, pantoprazole, and the like; antacids like magnesium carbonate, aluminium hydoxide, magnesium oxide and simethicone, and the like; cytoprotectives such as sucralphate, carbenoxolone sodium and misoprostol, and the like; antimuscarinic agents like pirenzepine, telenzepine and propanthelene bromide, and the like; drugs active against H. Pylori like bismuth salts such as bismuth subsalicylate, tripotassium dicitratobismuthate, ranitidine bismuth citrate, and the like; antibiotics for example clarithromycin, ofloxacin, levofloxacin, amoxycillin, and the like; all of which are suitable for use in the present invention.

Other medicaments that are suitable for this invention are drugs having solubility in acidic pH or ones having specific absorption sites in the upper part of the gastro-intestinal tract and those that are subjected to gastro-intestinal first pass metabolism (as in some reports stomach absorption is known to bypass gastrointestinal first pass metabolism) include antihypertensive agents like verapamil, nifedipine, propranolol, nimodipine, nicardipine, amlodipine, prazosin, ketanserin, guanabenz acetate, hydralazide, carvedilol, methyldopa, levodopa, carbidopa; antivirals like acyclovir, inosine, pranobex, zidovudine (AZT), tribavirin, vidarabine; lipid lowering agents like simvastatin, pravastatin, atorvastatin and lovastatin; antipsychotic agents like selegiline; sedatives like midazolam; all of which are suitable for use in the present invention.

The drug itself or its pharmacologically active salt or ester can be used in the present invention. Moreover, combination of drugs that are typically administered together may be included as the drug component. According to the present invention, the pharmaceutical composition provides a biphasic release of the drug.

By biphasic release it is meant that the pharmaceutical composition delivers a drug phase controlled release profile characterized by a rapid initial release of the drug followed by a controlled rate of release. The initial pulse provides an immediate release that quickly attains the therapeutic plasma drug levels while the second pulse provides a delayed release and a controlled release of the drug that extends therapeutic plasma drug levels initially achieved by the first pulse for a total prolonged period of time.

The immediate phase of the release profile may be defined as that portion of the drug that is released within about 30 minutes preferably within about 20 minutes, more preferably within about 10 minutes, after ingestion from the buoyant drug delivery system allowing the blood levels to quickly elevate to effective drug concentrations.

The controlled phase of the release profile may be defined as that portion of the drug that is released after about 45 minutes from the delivery system and that maintains the blood levels for extended periods of time.

Accordingly, about 5% to 60% and preferably about 10% to about 50% by weight of the total amount of drug is released immediately whereas about 40% to about 95% and preferably about 50% to about 90% by weight of the total amount of drug is released at a controlled rate.

In accordance to the invention, the total amount of drug is quantity by weight of the drug comprised in the whole pharmaceutical composition, a part of which is released immediately and remaining part is released at a controlled rate.

The total amount of drug is that which is typically administered for a given period of time. Accordingly, the drug may be present in a total amount ranging from a pharmaceutically acceptable amount up to 35% by weight of the total weight of the composition.

Sugar

According to the present invention the pharmaceutical composition contains sugars which provide low density airy structure of the desired texture to the matrix. Sugars preferably comprise a pharmaceutically acceptable saccharide, including a monosaccharide, a disaccharide, or a polyhydric alcohol, and/or mixtures of any of the foregoing. Examples of sugars preferred for the present invention include sucrose, glucose syrup, corn syrup, crystalline fructose, fructose, lactose, dextrose, galactose, maltodextrin, maltose, and the like, sugar alcohols like sorbitol, mannitol, maltol, maltitol, xylitol, lactitol. In more preferred embodiments of the subject invention the sugar is glucose syrup either in the dried form or as a liquid. Sugars-may be used alone or in combination with other similar sugars to achieve suitable matrix properties. In one preferred embodiment, sugar which is available under the brand name Glucidex (Roquette, UK) may be used.

The sugar may be present in an amount from about 5% to about 90% preferably from about 20% to about 85% and more preferably from about 40% to about 75% by weight of the total weight of the composition.

Gas Generating Component

According to the present invention, the pharmaceutical composition contains a combination of thermolabile and thermostable gas generating agents which aid in the formation of porous, preferably honeycombed structure and enhances the buoyancy of the formulation. As the name suggests, the thermolabile gas generating agent produces gas upon exposure to high temperature (of about or less than 200° C.) during heating operation while the thermostable agent does not dissociate upon exposure to temperatures stated above and produce gas upon contact with gastric fluid. Examples of thermolabile gas generating agents that may be used in the present invention include sodium bicarbonate, sodium glycine carbonate, potassium bicarbonate, ammonium bicarbonate, sodium bisulfite, sodium metabisulfite, and the like. The thermostable gas generating agent interacts with an acid source triggered by contact with water or simply with gastric acid to generate carbon dioxide or sulphur dioxide that gets entrapped within the porous, preferably honeycombed matrix of the composition and improves its floating characteristics. An example of a thermostable gas generating agent is calcium carbonate and sulfites such as sodium sulfite.

In those embodiments of the present invention, where the pharmaceutical composition is in the form of a capsule or tablet, thermostable gas generating agents may be used alone or in combination with an acid source as a couple. The acid source may be one or more of edible organic acids, a salt of an edible organic acid, or mixtures thereof. Examples of organic acids that may be used as the acid source in the present invention include citric acid or its salts such as sodium citrate or calcium citrate, malic acid, tartaric acid, succinic acid, fumaric acid, maleic acid or their salts, and the like. The organic acid salts which may be used as the acid source in the present invention include, for example, a mono-alkali salt of an organic acid having more than one carboxylic acid functional group, a bialkali metal salt of an organic acid having more than two carboxylic acid functional groups, and the like.

The gas generating components may be present in amounts from about 1% to about 40% preferably from about 1% to about 35% and more preferably from about 1% to about 30% by weight of the total weight of the composition.

Auxiliary Components

Optionally, other conventional pharmaceutical excipients known in the art of formulation development such as diluents, release retarding agents, inert oils, binding agents, spheronising agents, lubricants, glidants, fillers, or mixtures thereof, may also be incorporated into the buoyant formulation of the present invention.

Diluents

According to the present invention, the pharmaceutical composition may comprise a diluent which is stable to heating operation and form a part of the porous, preferably honeycombed structure. The diluent that may be used in the present invention, belongs to the class of excipients recognised in the art of pharmaceutical compounding. In preferred embodiments of the present invention, diluent is starch. Examples of starches that may be used in the present invention include maize starch, rice starch, potato starch or wheat starch. Examples of other diluents include dibasic calcium phosphate, calcium sulfate, powdered cellulose, microcrystalline cellulose, and the like.

The diluent may be present in an amount from about 3% to about 50% by weight of the total weight of the composition, preferably from about 5% to about 40% and more preferably from about 7% to about 35% by weight of the total weight of the composition.

Release Retarding Polymers

The pharmaceutical composition according to the present invention may also contain polymers to retard the release of the drug. These polymers may be present within the matrix structure of the pellets or capsules/tablets or may be coated onto the composition or may be added in capsule presentations of the present invention in the powder form. The polymers obtained as aqueous dispersions may replace water as granulating agent in the pellet preparations. Solid polymers may be added directly into the powder blend.

The polymers used may be of the hydrophilic or the hydrophobic type or pH dependent or pH independent in nature. Examples of the polymers suitable for this invention include the polymers well known in the pharmaceutical art for their release retarding properties, for example, cellulose ethers as hydroxypropyl celluloses of different grades, hydroxyethylcellulose, methylcellulose, hydroxypropyl ethylcellulose carboxymethyl cellulose, sodium carboxymethyl cellulose, hydroxyethyl methyl cellulose; acrylic polymers which are obtained as aqueous dispersions like Eudragit NE30D, Eudragit RS30D, Eudragit RL30D, Eudragit L30D or available as powders such as Eudragit RSPO, Eudragit RLPO, Eudragit L10055 (all supplied by Rohm Pharma, Germany), ethyl cellulose as aqueous dispersion or in powder form. Examples of highly swellable polymers that may be used in the present invention include hydroxypropyl methylcellulose of different grades, xanthan gums, sodium alginate, and the like.

The release retarding polymers may also be selected from the class of natural gums as karaya gum, locust bean gum, guar gum, gellan gum, and the like.

The one or more release retarding agents from the same or two different classes may be present from about 0.3% to about 25%, preferably from about 1.0% to about 20% or more preferably from about 1.5% to about 15% by weight of the total weight of the composition.

Other Auxiliarv Components

According to the present invention, the pharmaceutical composition may further contain a therapeutically inert oil which is solid at room temperature but softens at higher temperatures, that is, around 50-80° C. The oil, if present, acts as a release retarding agent. The oil is preferably, a fully hydrogenated or partially hydrogenated vegetable fat or oil. Examples of oils that may be used in the present invention include partially or fully hydrogenated cottonseed oil, coconut oil, soyabean oil, palm oil, kernel oil, peanut oil, sunflower oil, and the like. The oils preferred for the present invention are mentioned in the United States Pharmacopoeia as type 1 hydrogenated vegetable oils. These oils may be used alone or in combination with other oils having the same characteristics.

The oil may be present in an amount from about 0.2% to about 50% preferably about 0.2% to about 45% and more preferably about 0.4% to about 35% by weight of the total weight of the composition.

The pharmaceutical composition in the form of beads may also include a binder to provide cohesiveness to the powder mass. The binders commonly known to the pharmaceutical art may be used in the present invention. Examples of the binders are pregelatinised starch, polyvinylpyrollidone, hydroxypropyl methylcellulose, sodium carboxymethyl cellulose, starch paste, gelatin, xanthan gum, acacia, guar gum, and the like.

The binder may be present in amounts from about 0.1% to about 15%, preferably about 0.2% to about 12% and more preferably about 0.5% to about 10% by weight of the final weight of the composition.

In addition to the above ingredients, pharmaceutical grade magnesium stearate or stearic acid, and the like as a glidant, talc, and the like as an anti-adherent and silicon dioxide or hydrogenated vegetable oil or sodium stearyl fumarate, and the like as a lubricant may be incorporated in the pharmaceutical composition according to this invention.

According to the present invention, the pharmaceutical composition is prepared either in the form of pellets, granules, beads or as matrix capsules/tablets. The pellet/beads can be prepared using the commonly known techniques for extrusion and spheronisation and also other granulation techniques. Spheronising agents are added to the composition to get uniform spherical granules or pellets. Commonly used spheronisation aids are microcrystalline cellulose (Avicel PH 101 of FMC Corpn. and Emcocel 50M or Emcocel 90M of Mendell), mixture of microcrystalline cellulose and sodium carboxymethyl cellulose (Avicel RC 591 of FMC Corpn.)

The spheronising agent may be present in amounts from about 1% to about 30% preferably from about 2% to about 20% and more preferably from about 4% to about 15% by weight of the final weight of the composition.

According to the present invention the capsule shell may be of a hard gelatin or a soft gelatin type. Furthermore, the capsules made of starch or hydroxypropyl methylcellulose may also be used.

The pharmaceutical composition in accordance to the present invention is coated with the drug substance which provides the initial pulse of the biphasic release. The coat comprises a drug, a film forming polymer and optionally other suitable ingredients for coating including channelling agents, lubricants, coloring agents, flavors and plasticizers.

The film-forming polymer may be any suitable water soluble polymer that is conventionally used in the art. The polymers which are amenable to the biphasic therapy utilizing the novel therapeutic delivery system of the present invention include any of those suitable oral administration without compromising the drug release over the stipulated duration of a conventional, immediate release formulation. Examples, include, but not limited to, hydroxypropyl methylcellulose, hydroxypropyl cellulose, hydroxycellulose, carboxymethylcellulose and the like, and mixtures thereof. The drug coat may optionally include other pharmaceutically acceptable excipients recognized in the art of pharmaceutical coating such as starch, lactose, polyethylene glycol and the like as a channelling agent, talc, colloidal silica, magnesium stearate and the like as lubricants which aid in anti-sticking properties and triethyl citrate glyceryl monostearate, glyceryl triacetate, acetyl triethyl citrate, triethyl citrate dibutyl phthalate, dibutyl sebacate ethylene glycol and the like as plasticizers that increase flexibility and toughness of the coat by internally modifying or solvating polymer molecules.

The present invention is illustrated by, but is by no means limited to, the following examples.

### EXAMPLE 1

This example illustrates the present invention in the form of capsule formulation using carvedilol as an active agent. Two representative pharmaceutical compositions are illustrated in Table 1.

TABLE 1

| | Representative | Representative |
|---|---|---|
| | Capsule 1 | Capsule 2 |
| INGREDIENT | % W/W | % W/W |
| Carvedilol | 9 | 9 |
| Microcrystalline Cellulose (MCC) | 6.8 | 6.8 |
| Dried Glucose Syrup | 67.7 | 67.1 |
| Ammonium bicarbonate | 3.6 | 3.6 |
| Xanthan Gum | 6.7 | 7.2 |
| Hydrogenated cottonseed oil (lubritab) | 0.6 | 0.6 |
| Colloidal silicon dioxide | 0.5 | 0.5 |
| Calcium carbonate | 5.1 | 5.1 |

All the ingredients were sieved through 250 μmesh (British Standard Sieve (BSS), 60) and were blended in a Low shear mixer for 30 minutes. The blend was filled in size-0 gelatin capsules. The average fill weight was 490 mg. The capsules were given heat treatment at 90° C. for 20-30 minutes, following which they were cooled to room temperature.

The capsules were tested for in-vitro drug release in 1000 ml dissolution media of 0.1N HCl containing 1% sodium lauryl sulphate. The USP apparatus 2 with paddle speed at 100 rpm was used for the study. Paddles were fixed at 4.5 cm away from the base of the vessel and baskets, capped at the open end, were used as sinkers. The samples of the media were withdrawn at prescheduled timings and assayed for carvedilol content spectrophotometrically. The dissolution results are recorded in Table 2.

TABLE 2

| | Representative | | Representative | |
|---|---|---|---|---|
| | Capsule 1 | | Capsule 2 | |
| | Drug Release Profile | | Drug Release Profile | |
| | Non- | | Non- | |
| | cumulative | Cumulative | cumulative | Cumulative |
| Time (hr) | (%) | (%) | (%) | (%) |
| 0-1 | 29.0 | 29.0 | 21.6 | 21.6 |
| 1-2 | 5.0 | 34.0 | 5.6 | 27.2 |
| 2-4 | 12.0 | 46.0 | 11.1 | 38.3 |
| 4-8 | 27.0 | 73.0 | 18.4 | 56.7 |
| 8-12 | 13.0 | 86.0 | 12.7 | 69.4 |
| 12-16 | 14.0 | 100.0 | 10.6 | 80.0 |

It has been determined that in a controlled-release oral drug dosage form of the present invention with carvedilol as the drug substance, the maximum peak concentrations of carvedilol, upon oral ingestion, are equal to or lower than those produced by an immediate release pharmaceutical composition, and area under the concentration-time curve is substantially equivalent to that of the immediate release pharmaceutical composition. The pharmakokinetic parameters for carvedilol once-a-day formulations made according to example 1 are given in Tables 3 & 4.

TABLE 3
Study 1 (n = 9)
Using Representative capsule 1 formulation of example 1
Single dose, Open label, randomized, balanced,
crossover study under fed condition

| | Cₘₐₓ | AUC 0-t | AUC 0-inf | |
|---|---|---|---|---|
| Product | (mcg/ml) | (μg · h/mL) | (μg · h/mL) | Tₘₐₓ (hr) |
| Test Carvedilol | 70.08 | 498.7 | 559.18 | 5.22 |
| XL 50 mg OD | | | | |
| Reference (Coreg | 63.58 | 492.92 | 549.66 | 1.73 |
| 25 mg b.i.d.) | | | | |

TABLE 4
Study 2 (n = 8)
Using Representative capsule 2 formulation of example 1
Single dose, Open label, randomized, balanced, crossover
study under fed condition

| | Cₘₐₓ | AUC 0-t | AUC 0-inf | |
|---|---|---|---|---|
| Product | (mcg/ml) | (μg · h/mL) | (μg · h/mL) | Tₘₐₓ (hr) |
| Test Carvedilol | 46.2 | 319.97 | 360.35 | 5.09 |
| XL 50 mg OD | | | | |
| Reference | 55.28 | 377.05 | 408.51 | 1.56 |
| (Coreg 25 mg | | | | |
| b.i.d.) | | | | |

### EXAMPLE 2

This example illustrates the present invention in the form of tablet formulation using carvedilol as an active agent. The representative pharmaceutical composition is illustrated in Table 5.

TABLE 5

| | Representative Tablet |
|---|---|
| INGREDIENT | % W/W |
| Carvedilol | 10.61 |
| Microcrystalline Cellulose (MCC) | 6.63 |
| Dried Glucose Syrup | 62.33 |
| Ammonium bicarbonate | 4.64 |
| Xanthan Gum | 7.7 |
| Hydroxypropyl cellulose | 1.83 |
| Hydrogenated cottonseed oil (lubritab) | 0.61 |
| Colloidal silicon dioxide | 0.5 |
| Calcium carbonate | 5.1 |

All the ingredients were sieved through 250 μmesh (British Standard Sieve (BSS), 60) and were blended in a Low shear mixer for 30 minutes. The blend lubricated with sodium stearyl fumarate (1% w/w) and the tablets were compressed using suitable toolings. The tablets were given heat treatment at 90° C. for 20-30 minutes, following which they were cooled to room temperature.

The tablets were tested for in-vitro drug release in 1000 ml dissolution media of 0.1N HCl containing 1% sodium lauryl sulphate. The USP apparatus 2 with paddle speed at 100 rpm was used for the study. Paddles were fixed at 4.5 cm away from the base of the vessel and baskets, capped at the open end, were used as sinkers. The samples of the media were withdrawn at prescheduled timings and assayed for carvedilol content spectrphotometrically. The dissolution results are recorded in Table 6.

TABLE 6

Representative Tablet
Drug Release Profile

| Time (hr) | Non-cumulative (%) | Cumulative (%) |
|---|---|---|
| 0-1 | 9.2 | 9.2 |
| 1-2 | 4.2 | 13.4 |
| 2-4 | 11.9 | 25.3 |
| 4-8 | 25.8 | 51.1 |
| 8-12 | 14.2 | 65.3 |
| 12-16 | 11.5 | 76.8 |

Coating Compositions

The pharmaceutical compositions of example 1 and 2 are coated with the coating compositions illustrated in Table 7.

TABLE 7

| Ingredients | % W/W | | | |
|---|---|---|---|---|
| Carvedilol | 17 | 17.14 | 17.2 | 17.34 |
| Hydroxypropyl | 25.5 | 2.86 | 33.13 | 28.9 |
| methylcellulose | (5 cps) | (5 cps) | (6 cps) | (15 cps) |
| Hydroxypropyl cellulose | None | None | 33.13 | None |
| Polyethylene glycol | 2.83 | None | None | 8 |
| Povidone | 25.5 | 28.57 | None | None |
| Lactose | 25.5 | 28.57 | None | None |
| Cross povidone | None | 11.43 | None | None |
| Colloidal silicon dioxide | None | 1.9 | None | None |
| Titanium dioxide | None | None | 14.4 | 20.87 |
| Talc | 3.14 | None | None | None |
| Polysorbate 80 | 0.49 | None | None | None |
| Sod. Citrate dihydrate | None | None | None | 2.43 |
| Quinoline Yellow | None | None | 2.15 | None |
| Purified Water | q.s | q.s | q.s | q.s |

The components were added to water and stirred for about 60 min. The drug suspension was then homogenized and sprayed under standards set of conditions.

While the invention has been described by reference to specific examples, this was for the purpose of illustration only. Numerous alternative embodiments will be apparent to those skilled in the art and are considered to be within the scope of this invention.

What is claimed is:

1. A pharmaceutical composition which constitutes an oral controlled drug delivery system with biphasic release characteristics having a porous matrix, comprising:
at least one drug substance, sugar(s), and a gas generating component which is a combination of at least one thermostable and at least one thermolabile component, wherein the pharmaceutical composition further comprises a coating of said drug substance over said matrix such that the composition exhibits a biphasic release.

2. The pharmaceutical composition according to claim 1 wherein said pharmaceutical composition further comprises pharmaceutically acceptable auxiliary components.

3. The pharmaceutical composition according to claim 1 wherein the drug comprises at least one active compound selected from the therapeutic category of antiulcer, analgesic, antihypertensive, antibiotic, antipsychotic, anticancer, antimuscarinic, diuretic, antimigraine, antiviral, anti-inflammatory, sedatives, antidiabetic, antidepressant, antihistaminic, antiparasitic, antiepileptic, lipid lowering drugs, and mixtures thereof.

4. The pharmaceutical composition according to claim 1 wherein the drug is selected from the group consisting of enalapril, captopril, benazepril, lisinopril, ranitidine, famotidine, ranitidine bismuth citrate, diltiazem, propranolol, verapamil, carvedilol, nifedipine, acyclovir, ciprofloxacin, simvastatin, atorvastatin, pravastatin, lovastatin, selegiline, midazolam, glimepiride, glipizide, etodolac, nefazodone, and mixtures thereof.

5. The pharmaceutical composition according to claim 1 wherein the drug substance(s) is present in an amount ranging from a pharmaceutically acceptable amount up to about 35% by weight of said composition.

6. The pharmaceutical composition according to claim 1 wherein the sugar is a saccharide, a polyhydric alcohol, or a mixture thereof.

7. The pharmaceutical composition according to claim 6 wherein the sugar is selected from the group consisting of sucrose, glucose syrup, corn syrup, fructose, lactose, dextrose, galactose, maltose, maltodextrin, sorbitol, mannitol, maltol, maltitol, xylitol, lactitol, and mixtures thereof.

8. The pharmaceutical composition according to claim 1 wherein the sugar comprises about 5% to about 90% by weight of said composition.

9. The pharmaceutical composition according to claim 1 wherein the sugar comprises about 20% to about 85% by weight of said composition.

10. The pharmaceutical composition according to claim 1 wherein the sugar comprises about 40% to about 75% by weight of said composition.

11. The pharmaceutical composition according to claim 1 wherein the gas generating component is a sulfite, a carbonate or a bicarbonate salt.

12. The pharmaceutical composition according to claim 11 wherein the gas generating component is selected from the group consisting of ammonium bicarbonate, calcium carbonate, sodium bicarbonate, potassium bicarbonate, sodium glycine carbonate, sodium sulfite, sodium bisulfite and sodium metabisulfite.

13. The pharmaceutical composition according to claim 1 wherein the gas generating component comprises a gas couple comprising a thermostable gas generating salt and an edible organic acid or a salt of an edible organic acid.

14. The pharmaceutical composition according to claim 13 wherein the edible organic acid is selected from the group consisting of citric acid, ascorbic acid, tartaric acid, succinic acid, fumaric acid, malic acid, maleic acid, glycine, sarcosine, alanine, taurine and glutamic acid.

15. The pharmaceutical composition according to claim 1 wherein the gas generating component comprises about 1% to about 40% by weight of said composition.

16. The pharmaceutical composition according to claim 1 wherein the gas generating component comprises about 1% to about 35% by weight of said composition.

17. The pharmaceutical composition according to claim 1 wherein the gas generating component comprises about 1% to about 30% by weight of said composition.

18. The pharmaceutical composition according to claim 2 wherein the pharmaceutical auxiliary component comprises diluents, release retarding agents, inert oils, binding agents, spheronising agents, lubricants, glidants, fillers, or mixtures thereof.

19. The pharmaceutical composition according to claim 18 wherein the diluent is selected from the group consisting of starch, starch derivatives, cellulose derivatives, dibasic calcium phosphate and calcium sulfate.

20. The pharmaceutical composition according to claim 18 wherein the diluent is starch.

21. The pharmaceutical composition according to claim 20 wherein the starch is selected from the group consisting of maize starch, rice starch, potato starch and wheat starch.

22. The pharmaceutical composition according to claim 18 wherein the diluent comprises about 3% to about 50% by weight of said composition.

23. The pharmaceutical composition according to claim 18 wherein the diluent comprises about 7% to about 35% by weight of said composition.

24. The pharmaceutical composition according to claim 18 wherein the release retarding agent is either incorporated into the matrix or coated onto said composition.

25. The pharmaceutical composition according to claim 18 wherein the release retarding agent is selected from the group consisting of cellulose ethers, acrylic polymers, natural gums, and mixtures thereof.

26. The pharmaceutical composition according to claim 25 wherein the cellulose ether is selected from the group consisting of hydroxypropyl methylcellulose, hydroxypropyl cellulose, hydroxyethyl cellulose, hydroxypropyl ethylcellulose, methylcellulose, ethyl cellulose, carboxymethyl cellulose, sodium carboxymethyl cellulose, hydroxyethyl methylcellulose, and mixtures thereof.

27. The pharmaceutical composition according to claim 25 wherein the acrylic polymer is selected from the group consisting of methacrylates, polyacrylates copolymers, and mixtures thereof.

28. The pharmaceutical composition according to claim 25 wherein the natural gum is selected from the group consisting of xanthan gum, karaya gum, locust bean gum, sodium alginate, guar gum, gellan gum, and mixtures thereof.

29. The pharmaceutical composition according to claim 18 wherein the release retarding agent comprises about 0.3% to about 25% by weight of said composition.

30. The pharmaceutical composition according to claim 18 wherein the release retarding agent comprises about 1.5% to about 15% by weight of said composition.

31. The pharmaceutical composition according to claim 18 wherein the inert oil comprises a partially or fully hydrogenated vegetable oil.

32. The pharmaceutical composition according to claim 18 wherein the inert oil is selected from the group consisting of partially or fully hydrogenated cottonseed oil, castor oil, coconut oil, kernel oil, palm oil, soyabean oil, peanut oil, and mixtures thereof.

33. The pharmaceutical composition according to claim 18 wherein the inert oil comprises about 0.2% to about 50% by weight of said composition.

34. The pharmaceutical composition according to claim 18 wherein the inert oil comprises about 0.4% to about 35% by weight of said composition.

35. The pharmaceutical composition according to claim 18 wherein the binding agent is selected from the group consisting of pregelatinised starch, polyvinylpyrollidone, gelatin, hydroxypropyl methylcellulose, sodium carboxymethyl cellulose, natural gums, and mixtures thereof.

36. The pharmaceutical composition according to claim 18 wherein the binding agent comprises about 0.1% to about 15% by weight of said composition.

37. The pharmaceutical composition according to claim 18 wherein the binding agent comprises about 0.5% to about 10% by weight of said composition.

38. The pharmaceutical composition according to claim 18 wherein the spheronising agent is microcrystalline cellulose or a mixture of micro-crystalline cellulose and sodium carboxymethyl cellulose.

39. The pharmaceutical composition according to claim 18 wherein the spheronising agent comprises about 1% to about 30% by weight of said composition.

40. The pharmaceutical composition according to claim 18 wherein the spheronising agent comprises about 4% to about 15% by weight of said composition.

41. The pharmaceutical composition according to claim 1 further comprising a bioadhesive polymer.

42. The pharmaceutical composition according to claim 1 further comprising a highly swellable polymer.

43. The pharmaceutical composition according to claim 1 being formed into a physical form selected from the group consisting of multiple or single unit pellets, beads, granules, soft gelatin shell capsules, hard gelatin shell capsules or tablets.

44. The pharmaceutical composition according to claim 43 wherein the form of pellets, beads or granules is coated with a pharmaceutically acceptable film forming polymer or a pharmaceutical excipient.

45. The pharmaceutical composition according to claim 43 wherein the capsule shell is made of gelatin, hydroxypropyl methylcellulose or starch.

46. A controlled-release oral drug dosage form for releasing carvedilol, said dosage form comprising a porous matrix with said carvedilol, that releases said carvedilol into gastrointenstinal fluid by the dissolution and diffusion of said carvedilol out of said matrix by said gastrointenstinal fluid, and that upon immersion in gastrointenstinal fluid releases about 50% or less of said carvedilol within about four hours and more than about 75% of said carvedilol within about 16 hours after such immersion.

47. The controlled-release oral drug dosage form according to claim 46, wherein said drug dosage form is selected from the group consisting of multiple or single unit pellets, beads, granules, soft gelatin shell capsules, hard gelatin shell capsules, wherein the said dosage form, upon immersion in gastrointenstinal fluid, releases about 50% or less of said carvedilol within about four hours and more than about 75% of said carvedilol within about 12 hours after such immersion.

48. The controlled-release oral drug dosage form for releasing carvedilol according to claim 46, wherein said dosage form is a matrix tablet, which upon immersion in gastrointenstinal fluid, releases about 50% or less of said carvedilol within about four hours and more than about 75% of said carvedilol within about 16 hours after such immersion.

49. A controlled-release oral drug dosage form for releasing carvedilol, said dosage form comprising a porous polymeric matrix with said carvedilol, so that upon oral ingestion, maximum peak concentrations of carvedilol are equal to or lower than those produced by an immediate release pharmaceutical composition, and area under the concentration-time curve is substantially equivalent to that of the immediate release pharmaceutical composition.
